(19) United States
(12) Patent Application Publication
(10) Pub. No.: US 2022/0305156 A1
(43) Pub. Date: Sep. 29, 2022

(54) GERMICIDAL LAMP SYSTEM WITH REDUCED OPERATING TIME AND EXTENDED KILL AREA AND LAMP LIFE

(71) Applicant: LEONARDO ROQUE, VALRICO, FL (US)

(72) Inventor: LEONARDO ROQUE, VALRICO, FL (US)

(21) Appl. No.: 17/684,122

(22) Filed: Mar. 1, 2022

Related U.S. Application Data
(60) Provisional application No. 63/155,480, filed on Mar. 2, 2021.

(51) Int. Cl. A61L 2/10; A61L 2/24

(52) U.S. Cl. CPC A61L 2/10; A61L 2/24; A61L 2202/14; A61L 2202/11; A61L 2209/111

(57) ABSTRACT

A germicidal lamp system using ultraviolet light produced from at least one or more than one xenon flashlamp under high gas pressure and operating at a high voltage where each, in a multiple set of lamps, of the lamps is flashing at an interleaving mode to preserve lamp life and to reduce the sanitizing exposure time while still achieving a high kill ratio of microbiota in the area exposed to the UVC light. Advantages of the disclosed germicidal flashlamp system is that it exhibits increases in UVC Peak Optical Energy, reduced Exposure Time, increased Flashlamp Life/Durability, and increased Radiation Coverage.

### CLAIM OF PRIORITY

This application claims priority to and the benefit of US Provisional Application with Ser. No. 63/155,880, filed on Mar. 2, 2021, with the same title, the contents of which are hereby incorporated by reference in its entirety.

### FIELD AND BACKGROUND OF THE INVENTION

This invention generally relates to germicidal lamp systems having a plurality of germicidal lamps. Pathogenic microorganisms are becoming increasingly resistant to antimicrobial pharmaceuticals and, thus, treating germicidal infections are getting more difficult to treat. As a consequence, thorough disinfection of surfaces and objects is becoming increasingly important as a preventive measure against exposure. Examples of disinfection applications include sterilization of surgical tools, food and pharmaceutical packaging, decontamination of fluid streams, and area/room decontamination (e.g., disinfection of surfaces and objects in hospital rooms or for agricultural operations). It is known that irradiation of ultraviolet (UV) light in the spectrum between approximately 200 nm and approximately 320 nm is effective in deactivating and, in some cases, killing microorganisms, giving cause for the use of ultraviolet light technology for disinfection applications. UV is most effective between wavelengths of 200-280 nanometers, where effectiveness of UV C exposure is a function of time and intensity. A broad range of microorganisms are grouped as follows: bacteria, virus, fungi, protozoa and algae. The DNA of these organism is affected by UV exposure as UV light penetrates the cell wall and rearranges the microorganism's DNA preventing reproduction. An organism that cannot reproduce is considered to be microbiologically “DEAD”. Amount of UV light necessary to kill a particular organism is measured in units of millijoules per centimeter square. For example:

| | Deactivation Dosage |
|---|---|
| Microorganism | (mJ/cm²) |
| Corynebacterium Diphtheria | 6.5 |
| Dysentery Bacilli | 4.2 |
| Staphococus epidermidisa | 5.8 |
| Streptococcus facaelis | 10. |
| Bacteriophage (E. Coli) | 6.5 |

In addition, listed below are the most dangerous pathogens found in hospitals:

| | Deactivation Dosage |
|---|---|
| Microorganism | (mJ/cm²) |
| Methicillin-resistant Staphylococcus aureus(MRSA): | 10 |
| Clostridium difficile (C. Diff) Spores: | 20 |
| Vancomycin-resistant enterococci (VRE): | 15 |
| Carbapenem-resistant Enterobacteriaceae (CRE): | 15 |

What is the UVC dose for killing or disabling the COVID-19 virus? Because the COVID-19 virus (SARS-CoV-2) is so new, the scientific community does not yet have a specific deactivation dosage. However, we know the dosage values for comparable viruses in the same SARS virus family are 10-20 mJ/cm² using direct UVC light at a wavelength of 254 nm. This dosage will achieve 99.9% disinfection (i.e., inactivation) under controlled lab conditions. According to the International Ultraviolet Association, it is generally accepted that a dose of 40 mJ/cm² of 254 nm light will kill at least 99.99% of “any pathogenic microorganism” under controlled lab conditions.

One area where product availability is affected and is in high demand due to COVID 19 challenges is in room/area decontamination systems, particularly in hospitals or clinics where there are unique challenges in dispersing light over a large area and, thus, altering UV propagation from such systems may hinder such an objective. Accordingly, it would be beneficial to develop germicidal lamp apparatuses having features and/or configurations of components which improve the propagation of germicidal light toward desired objects and/or regions of a room in order to improve disinfection efficiency of the apparatus. In addition, it would be beneficial to develop room/area decontamination systems which are more effective and more efficient than conventional room/area decontamination systems.

### SUMMARY OF THE INVENTION

Embodiments of germicidal lamps disclosed herein include a support structure comprising an upper base and a lower base vertically spaced from each other that include at least one elongated high pressure xenon lamp with opposing ends respectively coupled to the upper and lower bases. In a related embodiment, the device includes two high pressure xenon lamps parallel to each other and are each interposed longitudinally between the upper and lower bases. In yet another example embodiment, the device includes three high pressure elongate xenon lamps that are each interposed longitudinally between their own bases (could also be a larger common base and top cover plate) and that illuminate an area in an interleaving pattern or mode to improve lamp life as well as extend area exposure coverage as the lamps are operating at higher energy levels thereby reducing individual operating times. Hence, no unit appears to exist in the prior art that has the UVC Optical power, nor the potential large “KILL” area, nor the small mobile compact size disclosed herein. The teachings herein are applicable to multiple flashlamps greater than three lamps and in which the pulsed lamp interleaving mode can be implemented.

In one example embodiment, there is provided a germicidal lamp system including at least one germicidal flashlamp supported by a top and bottom plate, the flashlamp including high pressure xenon gas and adapted to emit ultraviolet light when energized. There is included a power circuit coupled to the germicidal flashlamp and a processor and a storage medium having program instructions which are executable by the processor for: activating the power circuit to operate the germicidal lamp, and activating the power circuit to operate the germicidal lamp, wherein the power circuit is adapted to increase the lamp's voltage from 800 volts to 1600 volts thereby increasing the UVC peak optical energy by more than fourfold, as the electrical energy is: E=½ CV2, and the increase in the plasma display temperature to greater than 15,000° K, which will shift the flashlamp's spectra towards the shorter wavelengths (UVC) thus exhibiting a corresponding additional increase in the UVC peak optical energy of the lamp.

In a related example embodiment, there is provided germicidal lamp system that includes two flashlamps, with associated power, triggering, capacitance and processing circuits, a first and second flashlamps configured to operate with each other in an interleaving mode to increase the UVC peak optical energy while decreasing the exposure time of an area surrounding the flashlamp to about one-half the exposure and run time of a single flashlamp, thereby increasing end of life for the multiple flashlamps. In another related embodiment a germicidal lamp system includes three flashlamps configured to operate with each other in an interleaving mode and increase the UVC peak optical energy, thereby further reducing the UV exposure time while increasing the end of life of the multiple flashlamps.

In yet another related embodiment, there is provided a germicidal lamp system having a flashlamp cage or frame mounting such that a top and bottom support plates and one or more stainless steel rods provide at least a 3-point mounting or cage arrangement about each of the flashlamps, thereby minimizing damage by any outside mechanical forces. The flashlamps are each configured to rest on an oversized hollow nest at the bottom plate and held by the anode cable attached to one of the vertical rods allows for a mounting free of any mechanical and temperature stresses and provides for maximum radiation coverage and almost occultation-free coverage with 360° in the Azimuth plane and >150° in the elevation plane. In yet another example embodiment there is provided a germicidal lamp system with an improved high voltage power supply configuration for energizing more than one flashlamp in an interleaving flashing mode.

### BRIEF DESCRIPTION OF THE DRAWINGS

Other objects and advantages of the invention will become apparent upon reading the following detailed description and upon reference to the accompanying drawings in which:

FIG. 1A is a table that provides various operating parameters and associated values for an inventive flashlamp and germicidal system;

FIG. 1B is a flashlamp configuration for use in one or more germicidal system embodiments disclosed herein;

FIG. 1C illustrates an example embodiment of a germicidal lamp system that produces pulses of ultraviolet light for sanitation according to the teachings herein;

FIGS. 2A and 2B illustrate front and top views of the inventive flashlamp cage components and connections in detail, respectively.

FIGS. 2C and 2D illustrate a base and a top plate view of the flashlamp fixture;

FIG. 2E illustrates the inventive assembled flashlamp cage assembly including a flashlamp according to the teachings herein;

FIG. 2F illustrates a block diagram of the inventive flashlamp cage assembly with a description of the theory of operation and discharge specification;

FIG. 2G illustrate the operating specifications of the inventive germicidal lamp system taught herein;

FIG. 3A illustrates a block diagram of a germicidal lamp system with a single flashlamp according to the teachings herein;

FIG. 3B illustrates a more detailed interconnect diagram of the germicidal system without the flashlamp;

FIG. 3C illustrates evolution of the high voltage power supply for the germicidal lamp system described herein;

FIG. 4A illustrates a block diagram of a second germicidal lamp system using two flashlamps according to the teachings herein;

FIG. 4B illustrates a front control panel of the housing enclosing the control and power circuit powering the germicidal lamp system;

FIG. 4C illustrates a rear panel of the housing enclosure of the power circuit;

FIG. 4D illustrates the operating specifications of the inventive two flashlamp germicidal lamp system taught herein

FIG. 4E is a table that illustrates optical energy levels, exposure times and distances with single and twin flashlamps according to the teachings herein;

FIG. 5A illustrates a 4-quadrant motion detector sensors incorporated at or near the top plate of the flashlamp cage; and

FIG. 5B illustrates a block diagram of the complete motion detector circuit according to the teachings herein.

FIG. 6 a prior art example of a germicidal lamp system.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DESCRIPTION OF THE PREFERRED EMBODIMENTS

Following are more detailed descriptions of various related concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

A number of germicidal lamp systems have similar components, hence for brevity and enablement some of them are described herein, with reference to U.S. Pat. No. 9,517,284, which is herein incorporated by reference in its entirety. A commonly used flashlamp which may be considered for the germicidal sources described herein is a xenon flashtube as it generates a broad spectrum of light from ultraviolet to infrared and, thus, provides ultraviolet light in the entire spectrum known to the germicidal (i.e., between approximately 200 nm and approximately 320 nm). In addition, a xenon flashlamp can provide relatively sufficient intensity in the spectrum which is known to be optimally germicidal (i.e., 220 nm and/or between approximately 260 nm and approximately 265 nm) and can generate an extreme amount of heat, which can further contribute to the deactivation and/or killing of microorganisms, as well as generating low levels of ozone to improve kill rates. Flashlamps used in the various embodiments described herein are not limited to Xenon and include other gases (depending on output light characteristics wanted) such as helium, neon, argon, krypton, nitrogen, oxygen, hydrogen, water vapor, carbon dioxide, mercury vapor, sodium vapor and any combination thereof. In some embodiments, various additives and/or other substances may be included in the gas/es.

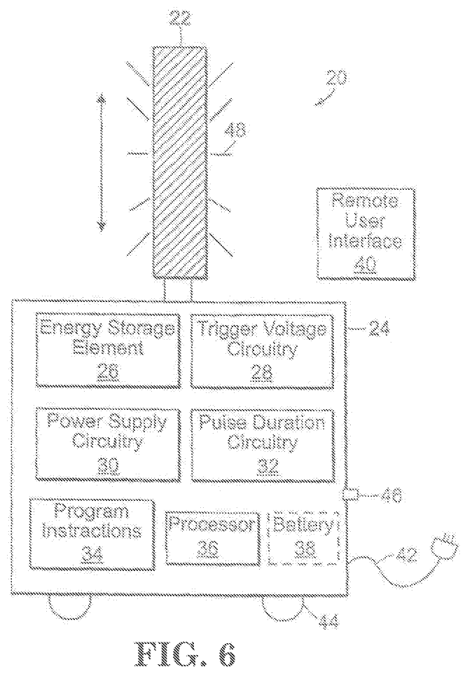

With reference to FIG. 6, a prior art germicidal apparatus 20 is shown with a base or base housing 24 with a number of components to affect such functionalities for germicidal pulsed light source 22, which includes energy storage element/s 26, trigger voltage circuitry 28, power circuitry 30, pulse duration circuitry 32, program instructions 34, processor 36, and optional battery 38. Optionally, apparatus 20 may include additional components, such as remote user interface 40, power cord 42, wheels 44 and occupancy sensor 46. It is noted that placement of the noted components are not restricted and may be disposed at any location to affect the functionality they impart to apparatus 20. Electrical components of apparatus 20 are in general in electrical communication with each other via wired and/or wireless connections to execute the operations of the germicidal apparatus. For instance, power circuitry 30 is electrically coupled to energy storage element/s 26, trigger voltage circuitry 28, and pulse duration circuitry 32 to generate a pulse of light from germicidal pulsed light source 22 and power circuitry 30 is further electrically coupled to processor 36, remote user interface 40 (and/or a user interface on the apparatus), and occupancy sensor 46 to affect the commencement and termination of operations of the apparatus. In addition, processor 36 is electrically and operatively coupled to program instructions and memory module 34 such that the program instructions may be executed by the processor and, in addition, processor 30 is operatively coupled to remote user interface 40 (and/or a user interface on the apparatus) and/or any sensors of apparatus 20 to execute the operations of germicidal pulsed light source 22 in accordance with program instructions 34.

Referring again to FIG. 6, trigger voltage circuitry 28 is configured to apply a sufficient voltage at a set frequency by which to activate pulsed germicidal light source 22 to generate recurrent pulses of light, while energy storage element/s 26 and pulse duration circuitry 32, respectively, are configured to discharge a set amount of stored energy in a set amount of time to pulsed germicidal light source 22. The components making up trigger voltage circuitry 28, energy storage element/s 26 and pulse duration circuitry 32 and the operation executed by such components will generally depend on design of the germicidal light source. For example, a flashlamp includes one or more capacitors for energy storage element/s and includes one or more inductors for its pulse duration circuitry 32. In addition, the trigger voltage in a flashlamp serves to ionize the gas in the flashlamp and cause the capacitor/s to discharge their accumulated energy thereto for the duration governed by the inductor/s. The voltage levels applied to trigger voltage circuitry 28 and pulse duration circuitry 32 as well as to energy storage element/s 26 to accumulate charge therein may generally depend on the design specifications of the germicidal apparatus and system (e.g., the desired pulse frequency, pulse duration, pulse intensity, exterior surface area of pulsed germicidal light source 22, among other parameters known to those skilled in the art of pulsed light source design). Germicidal apparatus 20 may include a user interface, such as a touch screen display or a keypad and, in some cases, a remote user interface 40, which includes but not limited to handheld communication devices (i.e., pagers, telephones, smartphones, smart devices etc.) and computers. User interfaces may also include but not limited to a start and stop button to enable a user to start and terminate an operation of apparatus 20, as well as touch sensors, audible means, and other graphical user interfaces or input controls allowing selection of different disinfection modes conducted by the germicidal apparatus.

The term “program instructions,” as used herein, refers to commands within software which are configured to perform a particular function and may be implemented in any of various ways, including procedure-based techniques, component-based techniques, and/or object-oriented techniques, among others. Program instructions 28 may be transmitted over or on a carrier medium such as a wire, cable, or wireless transmission link. In general, program instructions 28 may be stored with a storage medium within the apparatuses described herein. The term “storage medium”, as used herein, refers to any not limited to a read-only memory, a random access memory, or a magnetic or optical disk. Most, if not all of the aforementioned components are co-located in a single housing for protection of the individual components from the elements as well as for ease of portability.

One of the main purposes behind the design and development of the inventive germicidal UVC disinfectant systems (germicidal lamp 100) 200 and 400 described herein, is to provide a compact, mobile, clean, environmentally safe, and effective way to combat and kill all of the different types of pathogens that affect humanity. To that end, various advantages include: 1) a unique, simple, and efficient design of its High Voltage Power Supply; 2) use of a state of the art flashlamp that has higher Xenon gas pressure, higher anode Voltage and Peak current in excess of 1,000 amps, thereby providing a high intensity source of UVC radiation in the range from 190 to 280 nanometers; 3) a flashlamp housing (cage or mounting system) that is designed using a simple, yet sturdy, 3-point mounting design, thereby achieving a complete 360 degrees Azimuth coverage with minimum occultation, and maximizing the Elevation plane coverage (>150 degrees). Also, its construction of acrylic plastic and other UV radiation resistant materials, provides for a mount that is impervious to UV damage. The flashlamp cage has also 2 additional features: it provides for a stable vertical mounting of the lamp with no hard brackets or mounting clips that could provide stress to the lamp's fragile silica-glass envelope, causing it to crack or break (as the lamp's >15,000 degrees Kelvin plasma does produce some small temporary deformation of its envelope. The second feature is the ease in replacing the lamp in the field by disconnecting two quick terminals and easing the lamp out of its “loose” holder/mount. The germicidal systems disclosed herein also include an exposure Timer that selects a train of pulses at a rate of 10 pulses per minute from 1 minute to 99 minutes exposure.

In one example embodiment, the germicidal system produced over 1,000 Amps of Peak current with a corresponding, noticeable smell of Ozone in the air, direct indication of strong UV radiation. The unit and the 3 prototypes that are being built right now, will be tested for radiation intensity in the Spectra range from 190 to 280 nanometers at a certified Laboratory, to empirically arrive at its Peak Optical pulse energy level (theoretically arrived to be >90 Joules). An important discovery is Peak current and its measured Energy density mjoules/cm2 as an important correlation between both parameters to determine “end of life” of the flashlamp.

In another embodiment, a larger cabinet was designed along with a unit that incorporates two flashlamp cages (Twin Towers), to provide for twice its UVC Optical Energy and/or twice the lamps' life. In one embodiment, the method will be to alternatively trigger one of the two flashlamps at a rate of 1 pulse every six seconds and the other, delayed by 3 seconds, producing alternative Interleaved Flashing of 3 seconds intervals and halving the exposure time. In a related embodiment, the lamps may be pulsed a short as once per 60 seconds or 60 pulses in 60 seconds and is not limited to 1 pulse per six seconds. In this embodiment, the unit's power supply is changed to a modular, highly efficient, switching power supply, incorporating 4-400 VDC/200 watts modules in series to achieve the desired 1,600 VDC. A 5^{th} power supply module will be used to achieve 2,000 VDC for increase Optical Energy Output.

In one example embodiment, there is incorporated a 4-Quadrant motion detector for 360 degrees coverage at 3 meters minimum, which produces a log 6 kill zone of 5 meters in radius in all exposed surfaces/air, with all indications pointing to this outcome. In this example embodiment, the germicidal system in its memory module includes program instructions to inhibit or terminate activation of a power supply circuit to the germicidal source upon detecting movement and/or occupancy in the area/room in which the apparatus is arranged. Additional program instructions utilizing information from a movement detection sensor and/or a room/area occupancy sensor are included as well.

With further reference to the inventive flashlamp lamp systems 200 and 400, as will be discussed more hereinbelow, will emit intense UVC radiation in the range from 190 to 280 nanometers, with the implemented flashlamp being a new generation higher pressure xenon filled unit working at anode voltages from 1,600 to 2,000 VDC and producing flashlamp currents well in excess of 1,000 amps. In a Pulsed mode operation, these conditions translate into much higher percentage of UVC produced (>15 to 25%) of total radiation spectra, much higher than that produced by previous methods and systems.

The primary mode of operation is mobile, but can be implemented with units that are intended to be wall or ceiling mounted, and the unit will be mounted on top of a stainless steel, hospital grade cart. Some of the features include: High Voltage Power Supply; High Energy Capacitor Bank; High UVC “State of the Art” flashlamp; Long life Flashlamp(s); Quick in the field replacement of flashlamp(s); 4-Quadrant Motion Detector; Flashlamp Current Monitors; Exposure Timer; Delayed Start-up; and HV Shut Down.

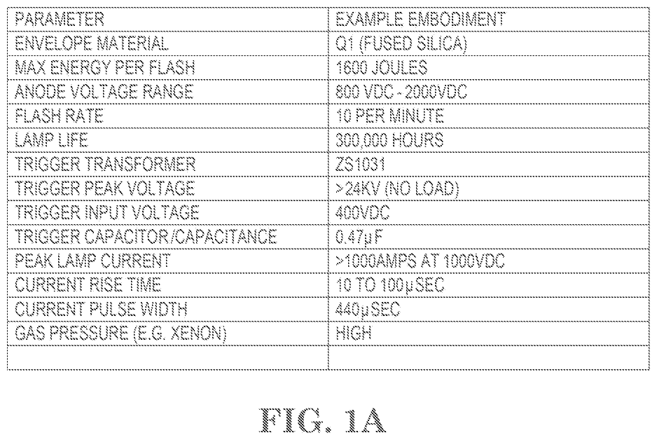

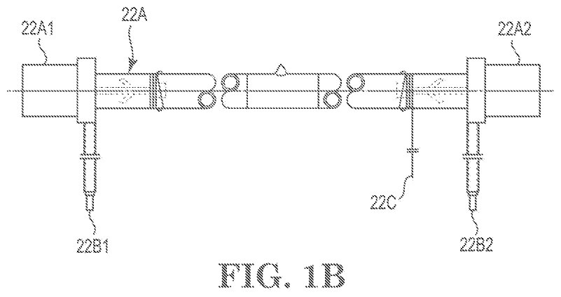

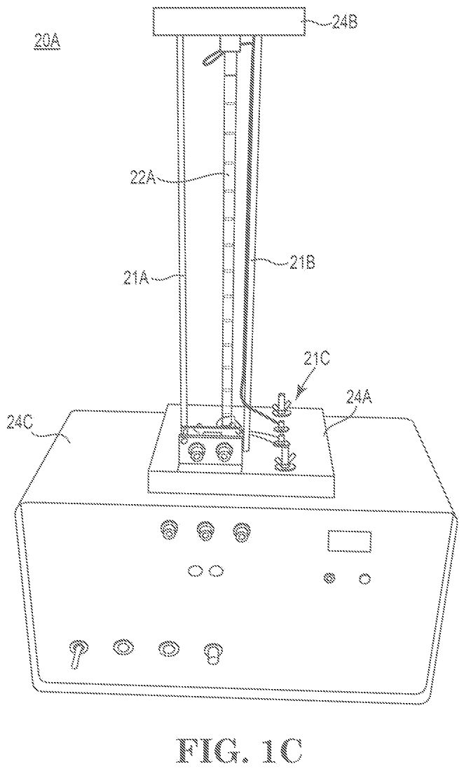

Referring now to the figures, FIG. 1A is a table that provides various operating parameters and associated values for an inventive flashlamp and germicidal system. FIG. 1B is a flashlamp configuration 22A for use in one or more germicidal system embodiments disclosed herein. Flashlamp 22A is made of Q1 fused silica that includes end caps 22A1 and 22A2, electrodes 22B1 and 22B2 and NiCr (Nickel/Chromium) wire 22C. In this example embodiment, the lamp is filled with xenon at high pressure to provide for more overall lamp life and pulsating performance.

Referring now to FIG. 1C illustrates an example embodiment of a germicidal lamp system 20A that produces pulses of ultraviolet light for sanitation according to the teachings herein. System 20A includes a flashlamp 22A mounted on a base 24A and a cover plate 24B supported by two metal rods 21A and 21B therebetween. Flashlamp 22A is further secured to base 24A via a series of wing nuts 21C and is supported by base housing 24C. Base housing 24C includes the trigger voltage circuitry, power supply circuitry, pulse duration circuitry, energy storage elements, processor and program instructions module as well as an input power module that energizes the base housing unit and its components as well as flashlamp 22A.

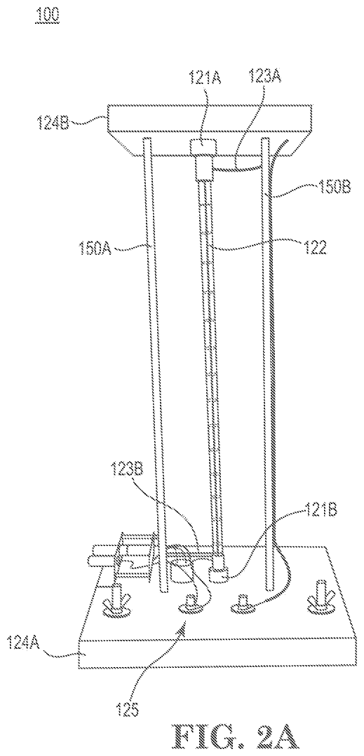

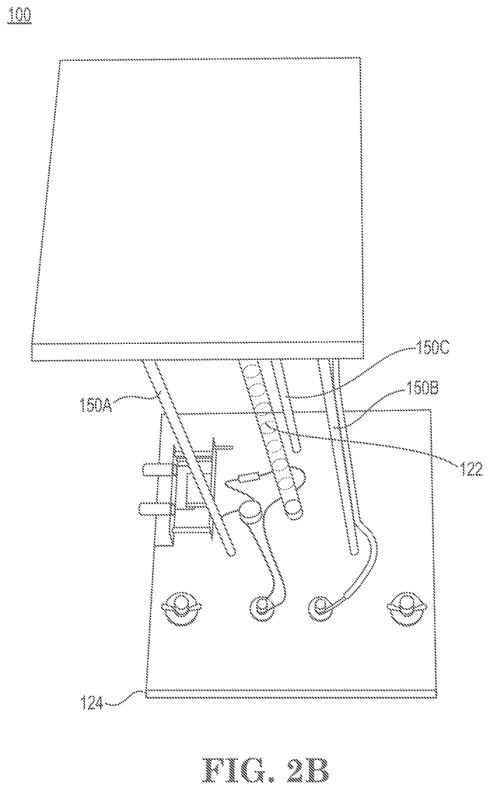

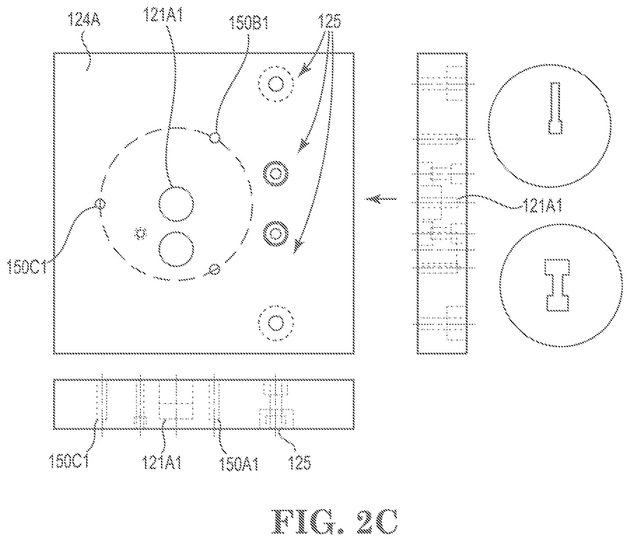

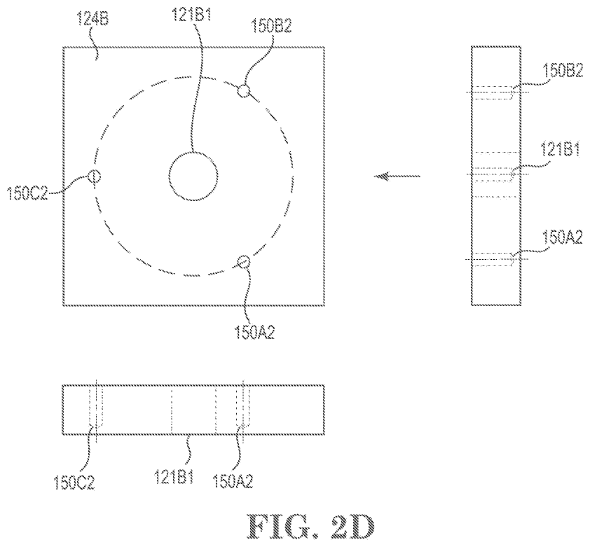

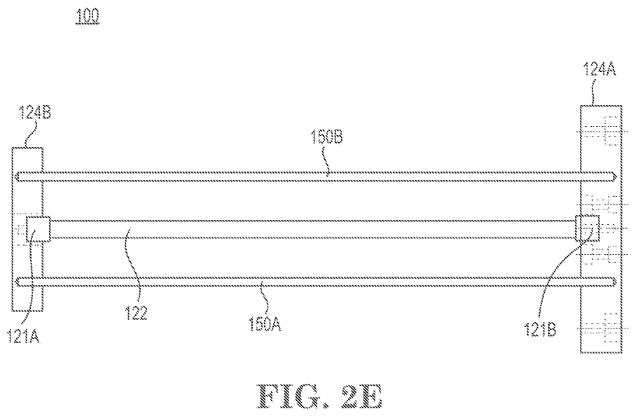

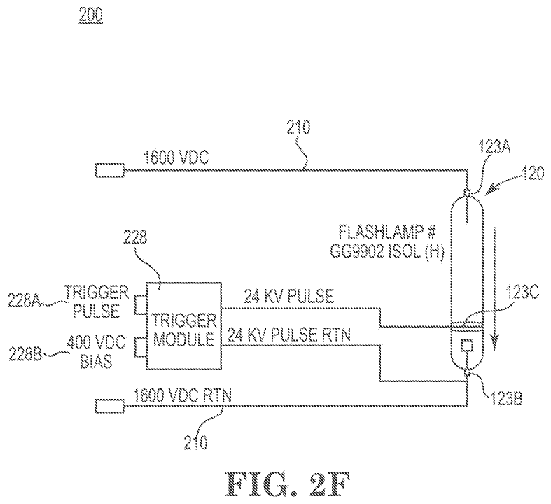

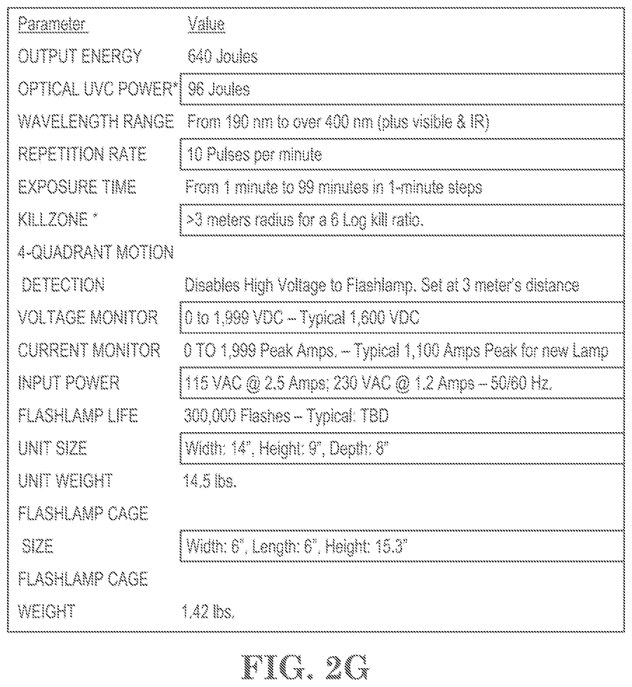

Referring now to FIGS. 2A-2G, an inventive flashlamp cage assembly and system 100 is described. In particular, FIGS. 2A and 2B illustrate front and top views of the inventive flashlamp cage components and connections in detail, respectively, while FIGS. 2C and 2D illustrate a base and a top plate, respectively, of the flashlamp fixture or cage 110 with the 3-point support mounting structure, which is the simplest and yet structurally sound support that has minimum radiation occultation. Additionally, the stainless steel connecting rods 150A-150C does provide reflection surfaces for the UVC radiation, thereby minimizing the small occultation problem. As can be ascertained, the Azimuth coverage is 360 degrees, and the elevation plane coverage surpasses 150 degrees in both hemispheres. As will be described further below, the 3-point structure and the soft mount of the flashlamp allows for operability and ease of maintenance. The preferred embodiment of germicidal lamp system 100 was built and tested, producing peak currents of 1,100 Amps. with a noticeable smell of Ozone, indicating high UV radiation levels. Certification of Kill Levels at 3 and 5 meters radius are possible with this embodiment.

Referring again to FIGS. 2A-2E, flashlamp assembly 100 includes a flashlamp or tube 122 interposed between base 124A and cover 124B and supported by 3 stainless steel rods 150A-150C. Lamp 122 include electrodes 123A and 123B, which are electrically coupled via conductive wires 123A1 and 123B1, respectively, to any one of connectors 125 on base 124A, which can then be electrically coupled to the base housing for electrification and control. Lamp endcaps 121A and 121B are configured to fit snuggly within corresponding center hole 121A1 in base 124A and center hole 121B1 in cover 124B as shown in FIGS. 2C and 2D, respectively. FIG. 2C illustrate top and front side and left side views of base 124A with center hole 121A1 and 3 holes (150A1-150C1) for stainless steel rods 150A-150C. FIG. 2D illustrate top and front side and left side views of base 124A with center hole 121A1 and 3 holes (150A1-150C1) for stainless steel rods 150A-150C. The bottom lamp end cap 121B rests in an oversized round cutout 151A1 and its top plate or cap (anode cap) 121A slips upwards longitudinally into and oversized open holes in each of the top 124B and bottom plates 124A, thereby allowing for ease of replacement and the lamp's anode 123A cable provides the flexible attachment to one of its connecting rods 150, thereby achieving a “soft” non-rigid mount design. This soft mount design, free of rigid mounts like clips, provides a safe, and free from mechanical and thermal stresses that could result in breakage of the lamp's fragile silica glass envelope. The 360 degrees Azimuth coverage is apparent in the design and its relatively small top plate, allows for a high angle elevation plane coverage (>150 degrees for both hemispheres). The flashlamp cage (tower) unit mounts to the top of Control Unit 124C cover using wing nuts 125 that allows for hand tightening. FIG. 2E illustrates a cutaway view of assembled flashlamp cage assembly 100 including a flashlamp according to the teachings herein.

Referring now to FIG. 2F illustrates a block diagram of the inventive flashlamp germicidal system 200 using flashlamp cage assembly 120 with a description of the theory of operation and discharge specification while FIG. 2G illustrates the operating specifications of the inventive germicidal lamp system 200 having an initial setting for a 3 meter exposure radius, at a pulse rate of about 10 pulses per minute, operating at a voltage of 1600 VDC and a 1000 amps of current, and capable of providing 300,000 flashes or pulses of light. In particular, flashlamp assembly 100 is powered via a 1660 VDC voltage source 210 (not shown) that is across anode 123A and cathode 123B such the current flows through the flashlamp (see arrow). Wire 122C receives a 24 KV pulse from a trigger module 228, trigger module 228 having inputs trigger pulse 228A and a 400 VDC voltage bias 228B. In this example embodiment, xenon is used as the discharge gas and gas within the flashlamp is at high pressure.

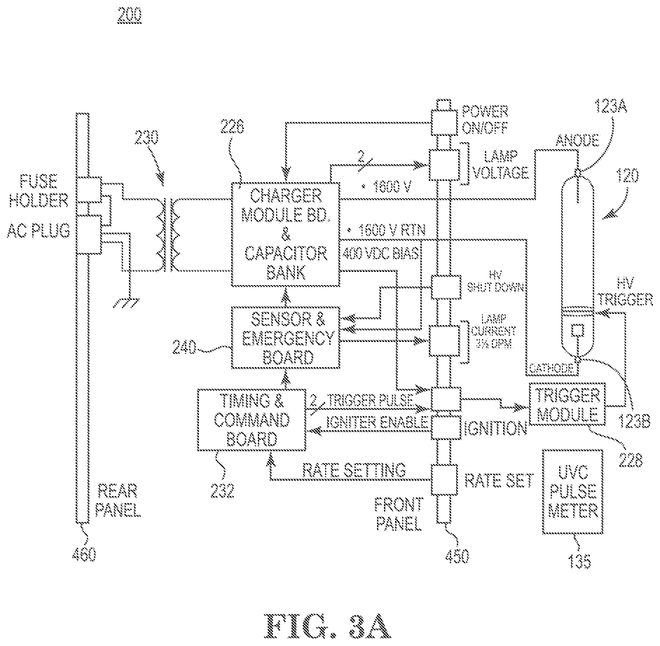

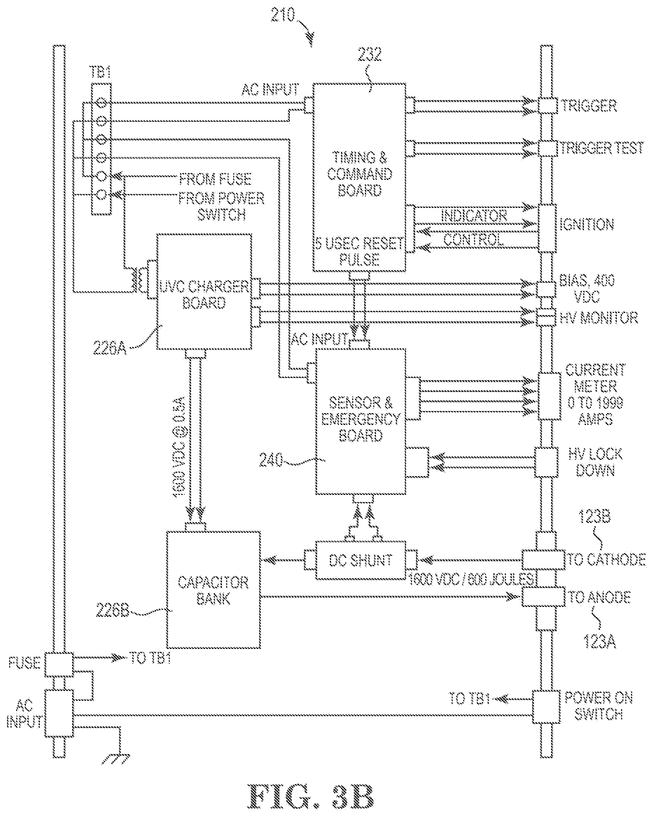

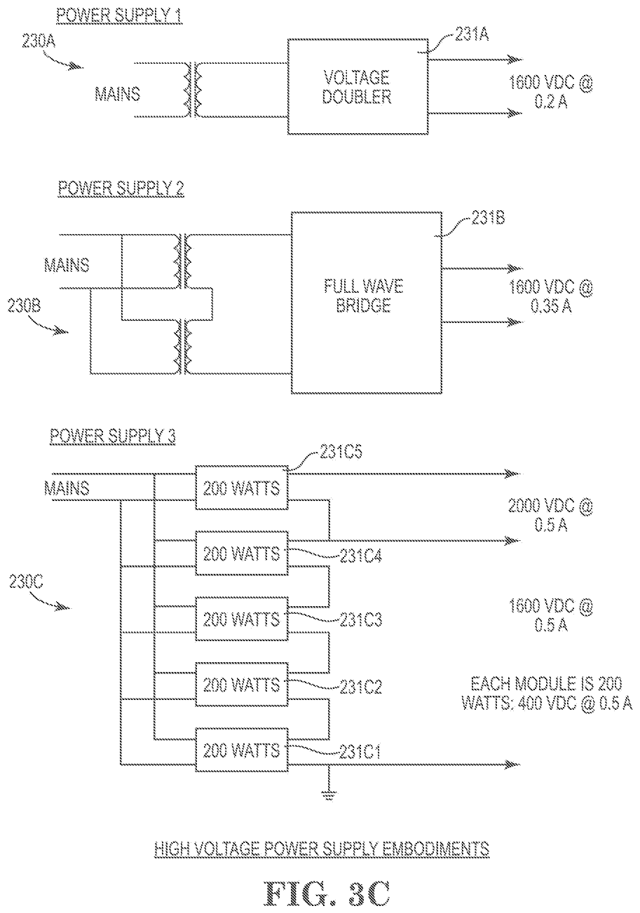

In this example embodiment and as illustrated in FIGS. 3A-3B, a block diagram of the inventive flashlamp or germicidal lamp system 200 includes a Power and Control Unit 210 electrically coupled to a flashlamp cage assembly 120. Power/Control unit 210 houses all of the necessary voltages and control signals to operate the Flashlamp 122. (FIG. 3B—Interconnect Diagram of the germicidal system without the flashlamp). Power/Control unit 210 includes a high voltage power supply 230 (depending on the embodiment, one or more Transformers), UVC Charger Board 226, a Timing/Command board 232, a Sensor/Emergency board 240, a Trigger Module 228 and a pulse meter 135. In one example embodiment, high voltage power supply 230A is comprised of a reversed connected control transformer feeding a voltage doubler circuit 231A to provide the 1600 VDC required. In a related embodiment, HV power supply 230B is a series connected dual reversed transformer driving a full bridge circuit 231B, as the extra power to feed two flashlamps (Twin Tower or dual lamp concept) is needed. HV power supply 230C includes four—200 watt power supply units (231C1-231C4) connected in series to provide 1600 VDC, with a fifth 200 watt power unit (23105) connectable with the previous four power units to provide 2000 VDC. As illustrated in FIG. 3C illustrates the various power supply designs 230A-230C provide power to multiple lamps and interleaving flashing schemes. Finally, the optimum High Voltage power supply will consist of 4, and later 5, series connected 200 watts modules, providing up to 800 watts of power at 1600 VDC and 1000 watts at 2000 VDC respectively. These modules are Power Factor Corrected (PFC) and fully isolated with an estimated efficiency of >90% each.

In this example embodiment, Energy Storage Module 226 is composed of 4-2000 μF/500 VDC capacitors in series (capacitor bank 226B) and a stabilizing circuit to provide equal energy sharing (charger board 226A). Its energy storage is E=½ CV²=½×500×10′×1600²=640 Joules. The amount of constant power to supply the unit is: Pt=E×Frequency=640×⅙ Hz=106 watts. The charging is done via a 2-1.5 K resistors, providing an RC time constant=500×10′×3000=1.5 secs., equivalent to a 98% charge at 6 seconds (4RC).

Referring now to the timing and command board 232, this board provides all of the timing and control signals for the germicidal lamp unit 122. Its control signals include: Trigger: 5 V Peak pulse, 600 usec wide, 10 pulses per minute repetition rate, duration controlled by Set Timer.

Reset: 5 V Peak pulse, 40 μsec wide, 10 pulses per minute repetition rate. Trigger Test: 5 V Peak pulse, 600 μsec wide, continuous 10 pulses per minute rate. Ignition: Momentary closure that starts the exposure time.

Referring now to the Sensor and Emergency Board 240, board 240 provides the interface to a 3½ inch digit digital panel meter that displays the flashlamp's peak current, by sensing the current through a 4 terminal Kelvin resistor of 0.0005 ohms and provides a display from 0 to 1999 Amps. It also provides the high voltage shutdown safety feature, activated by a front panel momentary switch to very quickly discharge the Energy Storage module 226 capacitors.

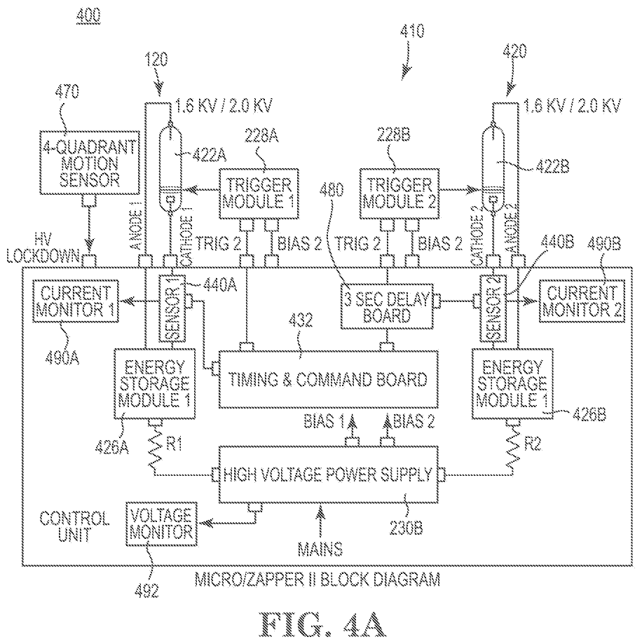

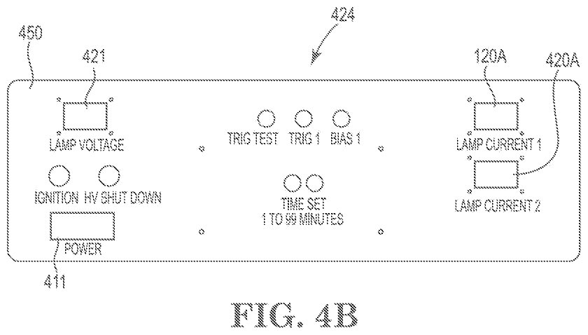

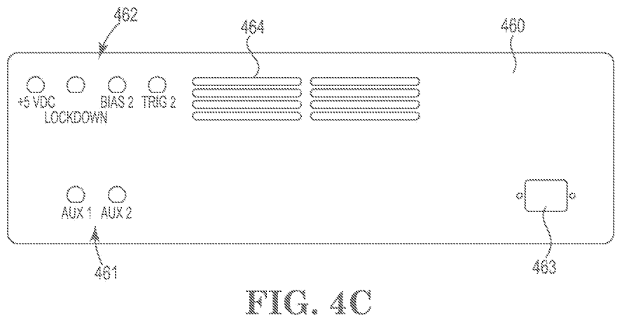

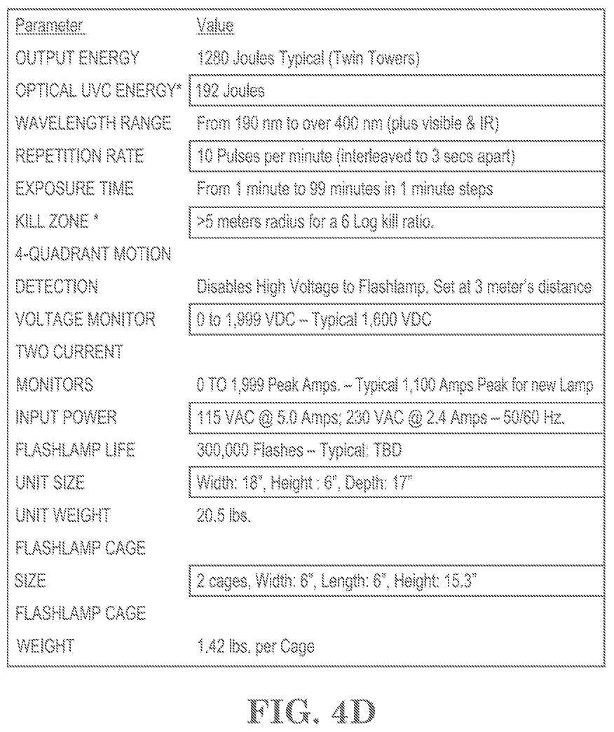

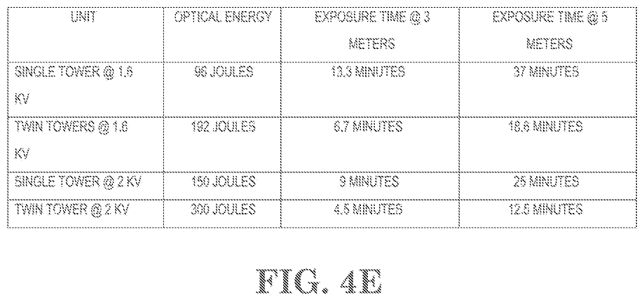

Referring now to FIGS. 4A-4E, there is illustrated another embodiment of the germicidal lamp disinfection system 400 and its various components. In particular, FIG. 4A illustrates a block diagram of a second germicidal lamp system 400 using two flashlamps according to the teachings herein. FIG. 4B illustrates a front control panel 450 of a housing 424 enclosing the control and power circuit 430 powering the germicidal lamp system 400, which include lamp current displays or lights 120A and 420A, a lamp voltage display or light 421 and a power button 411. FIG. 4C illustrates a rear panel 460 of the housing 424 enclosure of the power circuit 430, with auxiliary ports 461, various input jacks 462, a power port 463 and a vent 464. In this example embodiment, germicidal lamp system 400 includes two flashlamps 422A and 422B are utilized with an improved Power Supply (23B or 230C) for the additional load of 2 Energy Storage Modules and a larger cabinet. This embodiment also includes a 4-Quadrant Motion Sensor 470 mounted on top of one of the flashlamp assemblies (120 or 420). Another example embodiment of the germicidal lamp 400 includes a Control unit and two (2) Flashlamp Cage Assemblies 120 and 420. The Control Unit 410 has the following components Enhanced High Voltage Power Supply 230B that supplies 1600 VDC @ 0.35 Amps.; a Timing & Command Board 432 which supplies all of the control signals to operate the system. Two (2) Energy Storage Modules 426A and 426B that supplies the energy required by the Two (2) Flashlamps. Two Sensor/Emergency Boards 440A and 440B that provide the interface to two 3½ digit digital panel meter that displays the Flashlamp's peak currents. Peak currents are generated by sensing the current through a 4 terminal Kelvin resistor of 0.0005 ohms and provides a display from 0 to 1999 Amps. It also provides the High Voltage shutdown safety feature, activated by a front panel momentary switch to very quickly discharge the Energy Storage module's capacitors. A 3 sec. delay Board 480 for control signals to the 2^{nd} Tower (Interleave Mode). Two Digital Panel Meters (DPM) 490A and 490B for the two flashlamps Peak current monitoring. One DPM 492 to monitor the 1600 VDC from the HV Power Supply. Two high power resistors, R1 and R2 (200 watts) to charge the two Energy Storage Modules.

Referring now to FIG. 4E, and a discussion of germicidal lamp system calculations, there is a table that illustrates optical energy levels, exposure times and distances with single and twin flashlamps according to the teachings herein. In particular, the Table shows Exposure Time vs. Radius for the single and double Tower (or dual lamp) germicidal lamp, at 1.6 KV and 2.0 KV Anode voltage. For 640 Joules of Energy, 15% is available as UVC radiation out of the flashlamp used (Excelitas Input). Hence, the Optical Energy out of a single flashlamp is: 640×0.15=96 Joules. For 2 Towers (2 Flashlamps) The Optical Energy is =192 Joules (can extend/expand to 3 lamps). The Final Production Unit working at 2000 VDC Anode Voltage; the Optical Energy is:

Eo=½ CV²×0.15=½×500×10⁻⁶×2000²×0.15=150 Joules for 1 Flashlamp, and 300 Joules for 2 Flashlamps.
Area of a sphere 3 meters in radius: 4πr²=4×3.14×300²=1.13×10⁶ cm²
For 600 joules: Flux=600/1.13×10⁶=0.0005 J/cm²=0.5 mJ/cm²
If 15% of UVC* is available: Excelitas Input
Then: 0.5×0.15=0.075 mJ/cm² of UVC radiation @ 3 meters (per pulse)
For a kill of 6 log, need 10 mJ/cm²: (For the most hardy microorganism)
Therefore: 10/0.075=133 pulses @ 10 pulses/minute=13.3 minutes)
For an sphere of 5 meters radius, the number of pulses will be: 133×25/9=370 pulses for an exposure time of 37 minutes.
For the Twin Tower (2 flashlamps) unit, this time will half to 18.5 minutes,

In one example embodiment of a 2-flashlamp cage assemblies or “Twin Towers”, such obviates the small occultation problem and provides for a two-fold increase in the unit's UVC Optical Energy and/or a two-fold increase in each of the flashlamps' life, which results in reducing the exposure time to ½ the time. A larger cabinet provides for the additional electronics and adequate ventilation. The approach will be to alternatively trigger one of the two flashlamps at a rate of 1 pulse every six seconds and the other, delayed by 3 seconds, producing alternative Interleaved Flashing of 3 seconds intervals. The two flashlamps can also be worked to simultaneously be lit at a 1 pulse every 6 seconds. The unit's power supply will be changed to a modular, highly efficient, switching power supply, incorporating 4-400 VDC/200 watts modules in series to achieve the desired 1,600 VDC. A 5^{th} power supply module will be used to achieve 2,000 VDC for increase Optical Energy Output. The Twin Tower design could be expanded to multiple towers with a corresponding larger size, weight, and power consumption depending on the application.

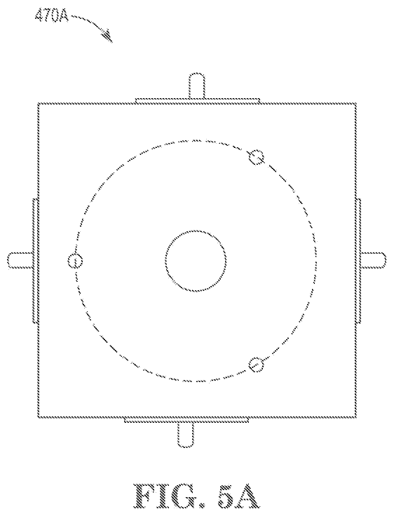

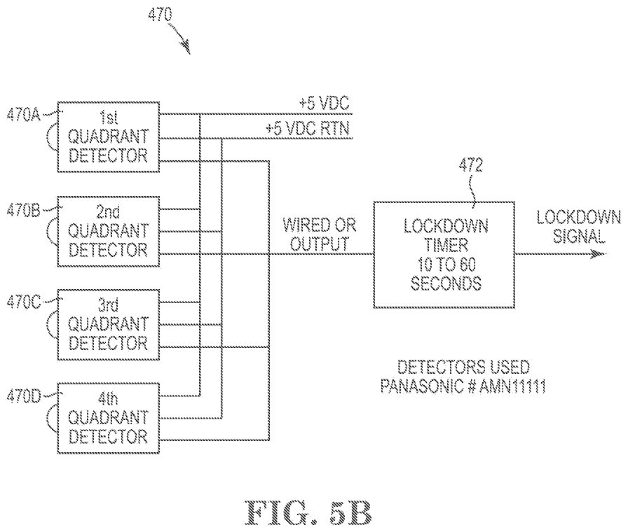

Referring now to FIG. 5A and FIG. 5B, FIG. 5A illustrates a 4-quadrant motion detector sensor 470A incorporated at or near the top plate of the flashlamp cage (either 120 or 420) while FIG. 5B illustrates a block diagram of the complete motion detector circuit 470a-470D all electrically or operatively coupled to a lockdown timer circuit 472 which is then coupled to system 200 or 400. In particular, 4 Quadrant Motion Detector circuit 470 includes 4 separate small boards mounted on one of the unit's Tower Top Plate. The small boards containing a fully functional Passive Infrared (PIR) motion detector from Panasonic, are mounted on each of the four sides of the Top Plate. They are fed by a +5 VDC supply and their outputs are tied together in a “wired or” configuration, so any detection from the detectors will actuate the “LOCK DOWN” feature in the germicidal lamp unit.

One of the main advantages of the lamp system UVC Disinfectant units 200 and 400 (germicidal lamp 100), is to provide a compact, mobile, clean, environmentally safe, and effective way to combat and kill all of the different types of pathogens that affect humanity. To this end, the germicidal lamp unit or system 200 will disinfect the air and surface micro-biota at a distance of about 5 meters to a log of about a 6 kill level at an exposure time of 18.7 minutes for a 10 mJ/cm² hardy microorganism (see Section 1.4—System Calculations). For spores this time could be doubled.

In one example embodiment, the germicidal lamp system 200 uses a 2000 VDC power supply to increase its UVC Optical Energy output to 300 Joules. The dual flashlamp design could be expanded to multiple towers with a corresponding larger size, weight, and power consumption, or 2 separate (or more) units could co-exist in an installation as the units' size will permit this scenario. This possibility looms large as the units' cost is relatively low. A larger stainless steel cart could house 2 of them for an impressive total Optical Energy of: 384 Joules for 1.6 KV operation or 600 Joules for 2.0 KV operation. Also, to reduce the shadowing effect (around corners, no line of sight), 2 separate units could be used at different locations within the encompassed area to be disinfected. To enhance portability, in one example embodiment the system uses a UPS with a 1 KVA rating battery bank, thereby providing about 8 hours of operation.

In another example embodiment, a portable Tri Tower System 300 (three flashlamps) is disclosed which provides when turned ON:
Set the Exposure time (1 to 99 minutes)
Upon actuating the Ignition switch, its indicator will turn on and, after a delay of 12 seconds, the unit will start working and producing Flashes separated and interleaved every 2 seconds until the desired exposure time has elapsed. At that point, the ignition indicator will turn off and the unit will cease to operate.
The current meter measures the lamps' peak current to confirm that they are working properly and determine their “End of Life”.
The voltmeter measures the unit's High Voltage Power Supply and is used as a diagnostic tool and to indicate when the Energy Storage capacitors have been discharged.
The HV Shut Down Pushbutton is used to discharge the Capacitors in the Energy Storage module.
The Backup Battery Power Station will operate the Tri Tower for over 3.2 straight Hours (5 hours for the Twin Tower)

Advantages of the disclosed germicidal lamp system design include increases in UVC Peak Optical Energy, Flashlamp Life/Durability, Radiation Coverage and reduction of exposure time. The increase of the lamp's voltage from 800 volts to 1600 volts increases the UVCPeak Optical Energy by more than fourfold, as the electrical energy is: E=½ CV², and the increase in the Plasma Display Temperature to greater than 15,000° K, will shift the lamp's Spectra towards the shorter wavelengths (UVC), and a corresponding additional increase (TBD) in the lamp's UVC Peak Optical Energy. The germicidal lamp novel HV Power Supply design and the new Flashlamp design are the main reasons for this claim. The use of 2 or 3 Flashlamp in an interleave mode provides the following advantages and in particular the Tri-Tower or 3 flashlamp embodiment: a) UVC Peak Optical Energy: It increases the peak optical energy threefold; b) Exposure Time: It decreases the exposure time to ⅓ of a single flashlamp; and c) Flashlamp Life: It increases the end of life of the flashlamps used 3-fold.

Finally, the flashlamp's mounting provides the following advantages: a) Flashlamp Life and Durability: the sturdy and yet simple 3-point mounting (cage), minimizes damage by any outside mechanical forces, and is impervious to UV radiation damage, as it is built with stainless steel and acrylic plastics. Its soft vertical attachment, resting on an oversized hollownest and at the top, held by the anode cable attached to one of the vertical ss rods (no hard brackets or mounting clips), allows for a mounting free of any mechanical and temperature stresses; and b) Radiation Coverage: The 3-point structure mount allows for almost occultation—free coverage (360° in the Azimuth plane and >150° in the elevation plane).
Flashlamp Maintenance—Another advantage is performance and End-of-Life monitoring of the flashlamps, needing to be established and certified, more so if the unit is used in a health facility. To this end, the peak current of the 2 or 3 lamps will be displayed (>1,000 Peak Amperes), and its reading logged in a Log Book. On initial installation, or when the flashlamp(s) need to be replaced, its initial Peak current will be logged in a Log Book (Performance). At end-of-day, or end-of-week intervals, readings of the lamp's current will be logged in the Log Book. Hence, when the readings fall to 80% of initial values (End of Life), the lamp should be replaced. Another advantage to the flashlamp mounting structure is the quick change out process when the lamp needs replacement when its peak current reading falls below 80% of its initial value. The simple and fast replacement of the lamp in the field includes disconnecting 2 terminals, ease the lamp out of its niche, replace it with a new lamp and connect the 2 terminals to the new lamp. The whole process shall take less than 5 minutes and then the user logs the new lamp initial current reading.

In one example embodiment, lamp's peak current will be sensed by an accurate 4-Terminal Kelvin resistor and fed to very low noise and high bandwidth operational amplifiers, capable of Peak Detecting the very fast rise time (<10 usec) of the current pulse. The current reading will decrease with use as the lamp's electrodes start corroding due to the extreme high temperatures of the Lamp's discharge (>15,000° K), most of the electrode's atoms will be deposited near the electrodes, but some will migrate to the center of the lamp and be deposited on the inside surface of the lamp's envelope, eventually reducing the UVC transmittance. There is a correlation between the reduction of the Lamp's Peak Current and its decrease in UVC Optical Output. This correlation will be determined empirically upon integration of an UVC Pulse Meter.

The following patents are incorporated by reference in their entireties: U.S. Pat. Nos. 8,816,301; 9,517,284; 10,245,340 and 10,874,760.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

1. A germicidal lamp system comprising:
at least one germicidal flashlamp having a fused silica envelope configured to contain a high pressure gas adapted to emit ultraviolet light when energized;
a high voltage supply adapted to provide 800 volts to 2000 volts to the at least one flashlamp;
a power circuit coupled to the germicidal flashlamp; and
a processor and a storage medium having program instructions which are executable by the processor, the processor adapted to activate the power circuit to operate the germicidal lamp and increasing the voltage on the at least one flashlamp from 800 volts to about 1600 volts thereby increasing output of the UVC peak optical energy.

2. The germicidal lamp system of claim 1 wherein the gas is xenon.

3. The germicidal lamp system of claim 1 wherein the increased UVC peak optical energy increases plasma display temperature to greater than about 15,000° K, thereby shifting the at least one flashlamp's spectra towards the shorter wavelengths or UVC range.

4. The germicidal lamp system of claim 1 wherein the further comprising a second flashlamp adapted to operate with the first flashlamp in an interleaving mode to increase the output of the UVC peak optical energy while decreasing the exposure time of an area surrounding the first and second flashlamps.

5. The germicidal lamp system of claim 4 further comprising a third flashlamp adapted to operate with the first and second flashlamps in the interleaving mode and increase the output of UVC peak optical energy and further reduce the UV exposure time, thereby increasing the end of life of multiple flashlamps.

6. The germicidal lamp system of claim 4 further including a 3-point mounting arrangement about each flashlamp adapted to provide some flexure for each flashlamp during operation while providing for maximum radiation coverage and almost occultation-free coverage with 360° in the Azimuth plane and >150° in the elevation plane.

7. The germicidal lamp system of claim 6 wherein the 3-point mounting arrangement includes top and bottom plates and one or more stainless steel rods mounted about each flashlamps, thereby minimizing damage by any outside mechanical forces, and wherein the flashlamp rests on an oversized hollow nest or aperture at the bottom plate and held by an anode cable attached to one of the vertical rods allows for a mounting free of any mechanical and temperature stresses.

8. The germicidal lamp system of claim 1 wherein the gas is selected from the group consisting of helium, neon, argon, krypton, nitrogen, oxygen, hydrogen, water vapor, carbon dioxide, mercury vapor, sodium vapor.

9. The germicidal lamp system of claim 4 wherein each of the two flashlamps are alternatively triggered at a rate of one pulse every six seconds and the second flashlamp being delayed by 3 seconds, thereby producing alternative interleaved flashing of 3 seconds intervals, the two flashlamps being operated to be lit at a one pulse every six seconds.

10. A germicidal lamp system comprising flashlamp system including a power and control circuit electrically coupled to a processor and a storage medium, the storage medium having program instructions which are executable by the processor, the system comprising:
at least two flashlamps with a high pressure fused silica envelope containing therein a gas under high pressure;
a high voltage supply adapted to provide 800 volts to 2000 volts to the at least two flashlamps; and
a trigger circuit for each flashlamp electrically coupled to the high voltage supply and to the processor and power circuit;
wherein the processor the power circuit to operate the germicidal lamps and increase a voltage of each flashlamp from 800 volts to 1600 volts, thereby increasing the UVC peak optical energy, and wherein the processor and the executable instructions operate the trigger circuit for each flashlamp in an interleaving mode.

11. The germicidal lamp system of claim 10 wherein the gas is xenon.

12. The germicidal lamp system of claim 10 further including a 3-point mounting arrangement about each flashlamp adapted to provide some flexure for each flashlamp during operation while providing for maximum radiation coverage and almost occultation-free coverage with 360° in the Azimuth plane and >150° in the elevation plane.

13. The germicidal lamp system of claim 10 further comprising a third flashlamp adapted to operate with the first and second flashlamps in the interleaving mode and increase the UVC peak optical energy, wherein the increased UVC peak optical energy further reduces the UV exposure time, thereby increasing the end of life of the multiple flashlamps.

14. The germicidal lamp system of claim 10 wherein each of the two flashlamps are alternatively triggered at a rate of one pulse every six seconds and the second flashlamp being delayed by 3 seconds, thereby producing alternative interleaved flashing of 3 seconds intervals, the two flashlamps being operated to be lit at a one pulse every six seconds.

15. A method of reducing exposure time and increasing flashlamp life in a germicidal multiple flashlamp system, the flashlamp system including a power and control circuit electrically coupled to a processor and a storage medium, the storage medium having program instructions which are executable by the processor, the method comprising the steps of:
configuring at least two flashlamps with a high pressure fused silica envelope containing therein a gas under high pressure;
providing a high voltage supply adapted to provide 800 volts to 2000 volts to the at least two flashlamps;
activating with the processor the power circuit to operate the germicidal lamps and increase a voltage of each flashlamp from 800 volts to 1600 volts, thereby increasing the UVC peak optical energy; and
triggering each flashlamp in an interleaving mode with the processor and the executable instructions.

16. The method of claim 15 further including the step of providing a third flashlamp adapted to operate with the first and second flashlamps in the interleaving mode and increase the UVC peak optical energy, wherein the increased UVC peak optical energy further reduces the UV exposure time, thereby increasing the end of life of the multiple flashlamps.

17. The method of claim 15 wherein the gas under high pressure is xenon.

18. The method of claim 15 further providing a 3-point mounting arrangement about each flashlamp adapted to provide some flexure for each flashlamp during operation while providing for maximum radiation coverage and almost occultation-free coverage with 360° in the Azimuth plane and >150° in the elevation plane.

19. The method of claim 18 wherein the 3-point mounting arrangement includes top and bottom plates and one or more stainless steel rods mounted about each flashlamps, thereby minimizing damage by any outside mechanical forces, and wherein the flashlamp rests on an oversized hollow nest or aperture at the bottom plate and held by the anode cable attached to one of the vertical rods allows for a mounting free of any mechanical and temperature stresses.

20. The method of claim 15 wherein the UVC optical energy is increased by at least more than fourfold thereby increasing plasma display temperature to greater than about 15,000° K resulting in a shift in the spectra of each flashlamp towards the shorter wavelengths or UVC range.
